(19) United States
(12) Patent Application Publication
(10) Pub. No.: US 2021/0024939 A1
(43) Pub. Date: Jan. 28, 2021

(54) DEVELOPMENT OF TECHNOLOGY FOR INDUCING OVER-EXPRESSION OF &#946;-AGARASE DAGA ENZYME

(71) Applicant: KOREA RESEARCH INSTITUTE OF BiOSCIENCE AND BIOTECHNOLOGY, Daejeon (KR)

(72) Inventors: Young Soo HONG, Daejeon (KR); So Heon SHIM, Daejeon (KR); I Seui PARK, Daejeon (KR)

(21) Appl. No.: 17/042,824

(22) PCT Filed: Mar. 25, 2019
(86) PCT No.: PCT/KR2019/003450

§ 371 Date: Sep. 28, 2020

(30) Foreign Application Priority Data
Mar. 26, 2018 (KR) 10-2018-0034518

(51) Int. Cl. C12N 15/67; C12N 15/76; C12P 19/14; C12P 19/60

(52) U.S. Cl. CPC C12N 15/67; C12N 15/76; C12Y 302/01081; C12P 19/60; C12P 19/14

(57) ABSTRACT

The present invention relates to a Streptomyces mutant strain in which a β-agarase DagA enzyme is over-expressed and a method for developing the strain. In addition, the present invention relates to a method for producing neoagarohexaose or neoagarotetraose in vivo by using the Streptomyces mutant strain.

### TECHNICAL FIELD

The present invention relates to a Streptomyces mutant strain capable of over-expressing a β-agarase DagA enzyme and a method for developing the same.

### BACKGROUND ART

Traditionally, agar or gelidium, which is dissolved in water and used for food, is a polysaccharide component that forms the cell wall of red algae, and generally consists of 40% agaropectin and 60% agarose.

The agar may be hydrolyzed to neoagarooligosaccharides (NAOs), which exhibit various effects such as whitening, moisturizing, antibacterial, and anti-inflammatory using chemicals or enzymes. Among the NAOs, neoagarohexaose and neoagarotetraose, which correspond to intermediates of agar, are reported to be particularly effective in fatigue recovery, anti-obesity, and anti-diabetes.

On the other hand, in the case of using a chemical method to prepare NAOs with various effects, there are time and cost benefits, but when hydrolyzing the NAOs through a chemical method, it is difficult to be directly applied to the pharmaceutical and cosmetic industries due to the problem that 5-(hydroxymethyl)furfural (5-HMF) which is a poisonous material is produced together.

Therefore, in order to overcome the above problems, a method of producing NAOs using a hydrolytic enzyme has been used instead of a chemical method. Specifically, enzymes used to hydrolyze the agar include α-agarase (EC 3.2.1.158) as an enzyme that cleaves α-1,3 bonds of the agar, and β-agarase as an enzyme that cleaves β-1,4 bonds thereof. The α-agarase may produce agarooligosaccharides in which α-1,3 bonds are cleaved, and in the case of hydrolyzing the agar using the β-agarase, the β-agarase may produce neoagarooligosaccharides in which β-1,4 bonds are cleaved.

Meanwhile, the β-agarase used for the hydrolysis is discovered even in Streptomyces coelicolor A3(2) which is a soil microorganism as well as marine-derived microorganisms such as Psedoalteronomonas, Alteromonas, Micrococcus, Vivrionaceae, etc.

The β-agarase present in the Streptomyces coelicolor A3(2) consists of DagA, DagB, and DagC enzymes (FIG. 1), and genes encoding the three enzymes are arranged in clusters. The DagA enzyme degrades agarose into neoagarohexaose (hereinafter referred to as ‘NA6’) and neoagarotetraose (hereinafter referred to as ‘NA4’), and is encoded by a sco3471 gene, has 309 amino acids (expected molecular weight of 35 kDa), and has a size of 32 kDa when secreted outside the cells. In addition, the DagB enzyme degrades NAOs into neoagarobiose or degrades the NA4 and NA6 produced by the DagA into neoagarobiose, and is encoded by a sco3487 gene. In addition, it is estimated that the DagC enzyme has a function to be degraded into D-galactose and 3,6-anhydro-L-galactose (3,6-ANG) as monosaccharides when the neoagarobiose produced by the DagB is absorbed into cells.

As illustrated in FIG. 2, the enzymes finally degrade the agar into neoagarobiose which is a form capable of being absorbed into the cells. Accordingly, in order to obtain only neoagarohexaose and neoagarotetraose, there is a limitation to perform the hydrolysis reaction of the agarose in vitro by purified the DagA enzyme from the Streptomyces coelicolor A3(2).

Therefore, in order to improve the production efficiency of neoagarohexaose and neoagarotetraose using a β-agarase enzyme in vitro, researches on a technology capable of obtaining the DagA enzyme in high yield, and furthermore, a technology capable of very efficiently producing neoagarohexaose and neoagarotetraose in vivo rather than in vitro are still needed.

### DISCLOSURE

### Technical Problem

The present invention is to provide Streptomyces mutant strains in which a β-agarase DagA enzyme is over-expressed and a method for producing the same.

Further, the present invention is to provide a method for producing a β-agarase DagA enzyme using the strain.

Further, the present invention is to provide a method for producing neoagarohexaose or neoagarotetraose in vivo without isolation and purification process of a β-agarase DagA enzyme.

### Technical Solution

In order to achieve the above objects, an aspect of the present invention provides a Streptomyces mutant strain over-expressing a β-agarase DagA enzyme in which the activity of a sco3485 gene and/or a sco3487 gene is reduced or lost.

Another aspect of the present invention provides a method for manipulating the Streptomyces mutant strain.

Yet another aspect of the present invention provides a method for producing a β-agarase DagA enzyme comprising the steps of incubating the Streptomyces mutant strain, and isolating a β-agarase DagA enzyme from the incubated Streptomyces mutant strain.

Still another aspect of the present invention provides a method for producing neoagarohexaose or neoagarotetraose in vivo comprising incubating a Streptomyces mutant strain, in which the activities of a sco3485 gene and a sco3487 gene are reduced or lost, in a medium containing agar.

### Advantageous Effects

According to the present invention, since the Streptomyces mutant strain of the present invention over-expresses the β-agarase DagA enzyme, it is possible to obtain the β-agarase DagA enzyme in high yield by using the Streptomyces mutant strain. In addition, when using the Streptomyces mutant strain of the present invention, it is possible to improve the production efficiency of neoagarohexaose or neoagarotetraose by producing neoagarohexaose or neoagarotetraose in vivo without isolation and purification process of an enzyme.

### DESCRIPTION OF DRAWINGS

FIG. 1 is a diagram showing a schematic diagram of a β-agarase gene cluster of Streptomyces coelicolor.

FIG. 2 is a diagram illustrating an exploded schematic diagram of agarolytic pathway.

FIGS. 3(A) to (C) are schematic diagrams of a vector for reducing or losing the activity of a sco3485 gene and a diagram illustrating a result of confirming the size of a vector prepared through polymerase chain reaction (PCR).

FIGS. 4(A) to 4(C) are diagrams illustrating results of performing PCR and gene sequencing to confirm whether the activity of a sco3485 gene is reduced or lost in a mutant strain.

FIGS. 5(A) to 5(C) are schematic diagrams of a vector for reducing or losing the activity of a sco3487 gene and a diagram illustrating a result of confirming the size of a vector prepared through polymerase chain reaction (PCR).

FIGS. 6(A) to 6(C) are diagrams illustrating results of performing PCR and gene sequencing to confirm whether the activity of a sco3487 gene is reduced or lost in a mutant strain.

FIGS. 7(A) and 7(B) are diagrams illustrating results of performing PCR and gene sequencing to confirm whether the activities of both sco3487 and sco3485 genes are reduced or lost in a CRI85B strain.

FIG. 8 is a diagram illustrating results of confirming a substance reacting with agarose through a protein from a culture medium of a CRIDb strain through TLC chromatography.

FIGS. 9 to 11 are diagrams illustrating results confirmed by performing a zymogram assay to confirm phenotypes according to protein production by varying a carbon source contained in culture plates, respectively.

FIGS. 12(A) and 12(B) are diagrams illustrating results of confirming expression levels of a β-agarase DagA enzyme protein in CRIDb strain, CRI3485 strain, and CRI85B strain through Western blot analysis.

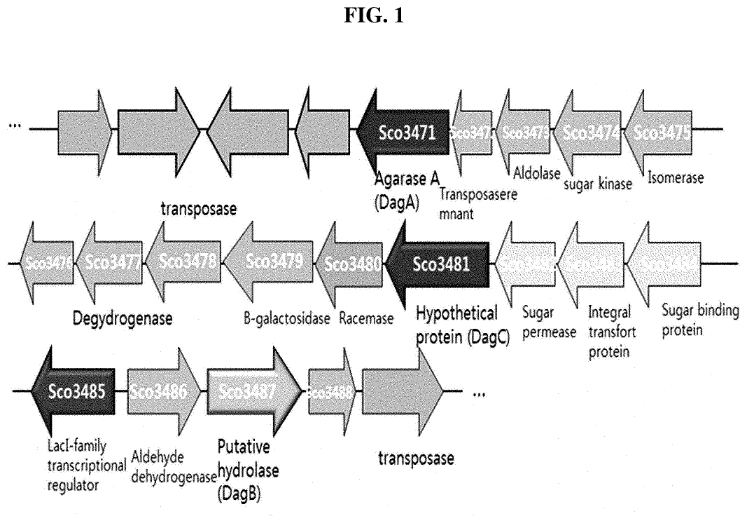

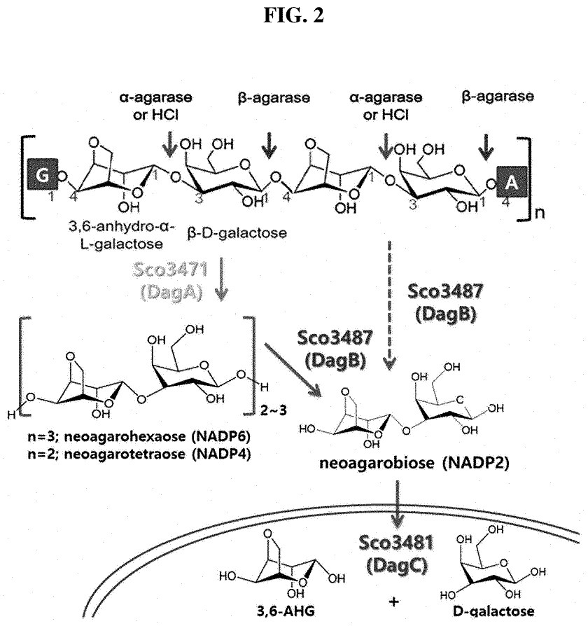

### BEST MODE

Hereinafter, the present invention will be described in detail.

1. β-Agarase DagA Over-Producing Streptomyces Mutant Strain and Method for Producing the Same

One aspect of the present invention provides a Streptomyces mutant strain for over-expressing a β-agarase DagA enzyme.

In the Streptomyces mutant strain for over-expressing the β-agarase DagA enzyme of the present invention, the activity of a sco3485 gene is reduced or lost.

The sco3485 gene is a gene encoding a protein that inhibits expression of β-agarase, particularly a gene encoding a DagA enzyme, and includes a nucleic acid sequence of SEQ ID NO: 1.

The reduction or loss of the gene activity may be achieved by inducing mutations according to substitution, deletion, insertion, or combinations thereof in the entire nucleic acid sequence or partial nucleic acid sequence of the sco3485 gene. The mutation of the gene may be achieved through a general method called knock-out or knock-down in the related art, and for example, may be achieved through gene editing technology such as CRISPR-Cas9.

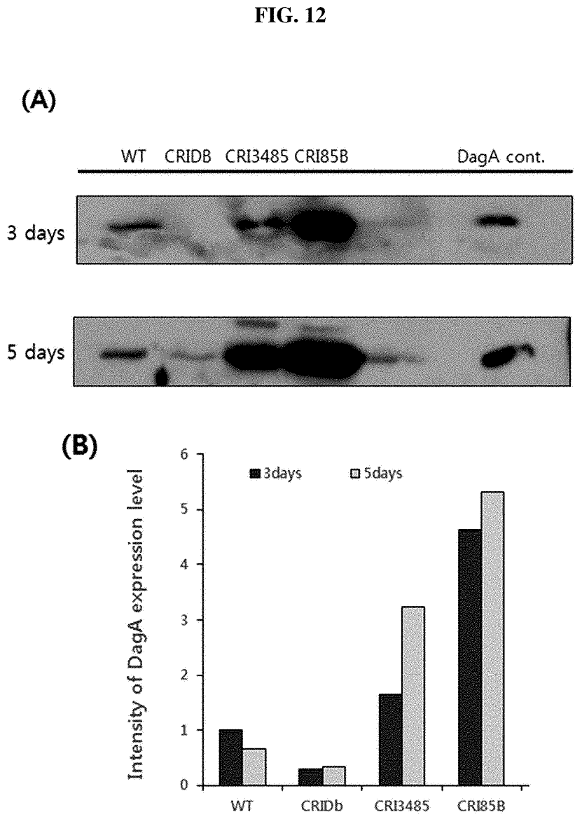

As described above, since the activity of the sco3485 gene is reduced or lost, the expression of the β-agarase DagA enzyme may not be inhibited, and as such, in the Streptomyces mutant strain, the β-agarase DagA enzyme is over-expressed. In a specific embodiment of the present invention, it was confirmed that the expression of the DagA enzyme was increased in a CRI3485 strain in which the activity of the sco3485 gene was reduced or lost (see FIG. 12).

In addition, in the Streptomyces mutant strain for producing the β-agarase DagA enzyme of the present invention, the activity of a sco3487 gene may be additionally reduced or lost.

The sco3487 gene is a gene encoding a β-agarase DagB enzyme, and includes a nucleic acid sequence of SEQ ID NO: 10.

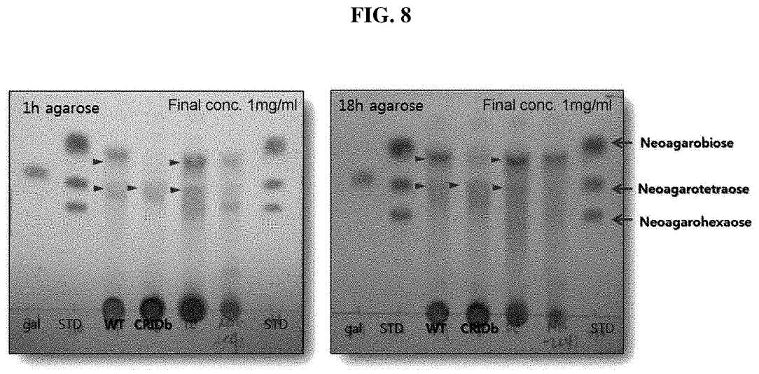

The β-agarase DagB enzyme encoded by the sco3487 gene degrades neoagarooligosaccharide into neoagarobiose in the Streptomyces strain, or degrades neoagarohexaose or neoagarotetraose produced by the β-agarase DagA enzyme into neoagarobiose. In a specific embodiment of the present invention, after a CRIDb strain having reduced or lost activity of the sco3487 gene was incubated in a medium containing agarose, components of the medium are analyzed using TLC chromatography, and as a result, it was confirmed that neoagarobiose was not produced (see FIG. 8).

The reduction or loss of the activity of the sco3487 gene may also be achieved by mutating the corresponding gene using a general method called knock-out or knock-down in the related art, and for example, may be achieved through gene editing technology such as CRISPR-Cas9.

In addition to the sco3485 gene as described above, in the Streptomyces mutant strain in which the activity of the sco3487 gene is further reduced or lost, the β-agarase DagA enzyme may be further over-expressed. In a specific embodiment of the present invention, it was confirmed that in the CRI85B strain in which the activities of the sco3485 gene and the sco3487 gene were reduced or lost, the expression of the DagA enzyme was more effectively increased compared to the CRI3485 strain (see FIG. 12).

Another aspect of the present invention provides a method for producing a Streptomyces mutant strain over-expressing a β-agarase DagA enzyme.

The producing method of the present invention includes reducing or losing the activity of the sco3485 gene in the Streptomyces strain.

The reducing or losing of the activity of the sco3485 gene may be achieved by inducing mutations according to substitution, deletion, insertion, or combinations thereof in the entire nucleic acid sequence or partial nucleic acid sequence of the sco3485 gene. The mutation of the gene may be achieved through a general method called knock-out or knock-down in the related art, and for example, may be achieved through gene editing technology such as a CRISPR-Cas9 system and the like.

The knock-out or knock-down for genetic mutation of the Streptomyces strain may be performed according to a suitable transgenic method known in the art to introduce a substance capable of reducing or losing the activity of a gene in cells, and for example, may be achieved through a transformation method such as E. coli conjugation.

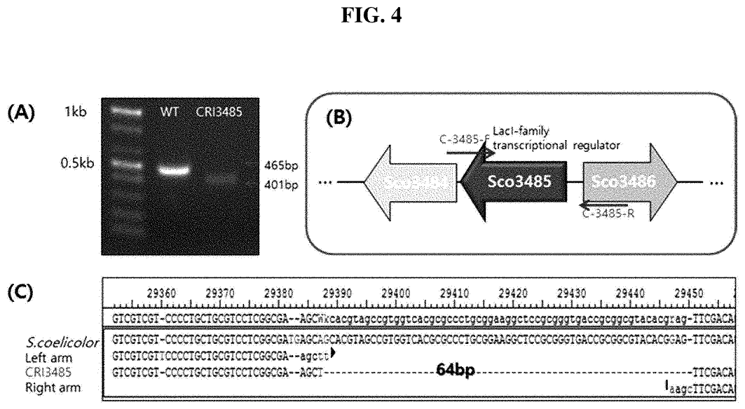

In the CRISPR-Cas9 system, a guide RNA that specifically binds to a target site of the gene recognizes a site of a target gene, and the guide RNA forms a complex with a Cas9 protein so that the Cas9 protein has endonuclease activity. Thereafter, homology-directed repair (HDR) is performed using a homologous nucleic acid sequence of which a part is deleted as a template strand, thereby achieving gene editing. In a specific embodiment of the present invention, it was confirmed that the activity of the sco3485 gene may be reduced or lost by deleting 64 bp of the sco3485 gene by using a dCRI3485 vector to which the CRISPR-Cas9 system is applied (see FIG. 4).

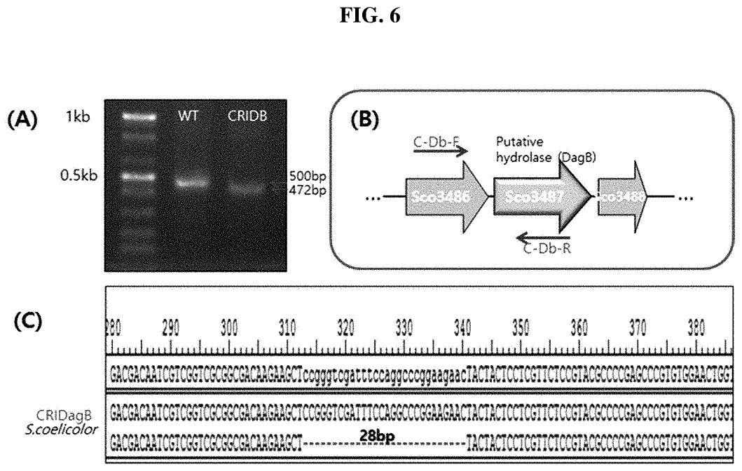

The producing method of the present invention further comprises reducing or losing the activity of the sco3487 gene. The reduction or loss of the activity of the sco3487 gene may be performed through knock-out or knock-down for gene mutation, and may be achieved through gene editing technology such as CRISPR-Cas9 described above, and thus, the detailed description thereof will be omitted. In a specific embodiment of the present invention, it was confirmed that the activity of the sco3487 gene may be reduced or lost by deleting 28 bp of the sco3487 gene by using a dCRIDb vector to which the CRISPR-Cas9 system is applied (see FIG. 6).

Meanwhile, the processes of reducing or losing the activities of the sco3485 gene and the sco3487 gene may be performed sequentially or may be performed simultaneously.

As described above, it is possible to produce the Streptomyces mutant strains in which the β-agarase DagA enzyme is further over-expressed by reducing or losing the activities of both the sco3485 gene and the sco3487 gene.

2. Method for Producing β-Agarase DagA Enzyme

Another aspect of the present invention provides a method for producing a β-agarase DagA enzyme.

The method for producing the β-agarase DagA enzyme of the present invention comprises the steps of incubating a Streptomyces mutant strain and isolating a β-agarase DagA enzyme from the incubated Streptomyces mutant strain, which has been described in “1. β-agarase DagA over-producing Streptomyces mutant strain and method for producing the same e” above.

In the Streptomyces mutant strains, since the β-agarase DagA enzyme is over-expressed, the β-agarase DagA enzyme may be effectively produced. The detailed description thereof will be omitted by citing the description of the item of “1. β-agarase DagA over-producing Streptomyces mutant strain and method for producing the same” above.

Since the Streptomyces mutant strain over-expresses the β-agarase DagA enzyme, it is possible to increase the production yield of the β-agarase DagA. In addition, when the activities of both the sco3485 gene and the sco3487 gene are reduced or lost, the expression of the β-agarase DagA enzyme is further increased, thereby making it possible to increase the production efficiency of the enzyme using the strain in which the activities of both the sco3485 gene and the sco3487 gene are reduced or lost.

The Streptomyces mutant strain may be incubated according to an appropriate medium and incubation conditions known in the art. Those skilled in the art may easily adjust and use the medium and incubation conditions according to a type of Streptomyces mutant strain to be selected. The incubation method may include a batch type, a continuous type, a fed-batch type, or a combination thereof.

The medium may contain various carbon sources, nitrogen sources, and trace element components.

The carbon sources may include, for example, carbohydrates such as glucose, sucrose, lactose, fructose, maltose, starch, and cellulose, fats such as soybean oil, sunflower oil, castor oil, and coconut oil, fatty acids such as palmitic acid, stearic acid, and linoleic acid, alcohols such as glycerol and ethanol, organic acids such as acetic acid, or combinations thereof. The incubation may be performed using glucose as the carbon source. The nitrogen sources may include organic nitrogen sources such as peptone, yeast extract, broth, malt extract, corn steep liquor (CSL), and soybean meal, inorganic nitrogen sources such as urea, ammonium sulfate, ammonium chloride, ammonium phosphate, ammonium carbonate, and ammonium nitrate, or combinations thereof. As a supply source of phosphorus, the medium may contain, for example, potassium dihydrogen phosphate, dipotassium hydrogen phosphate and sodium-containing salts corresponding thereto, and metal salts such as magnesium sulfate or iron sulfate.

In addition, amino acids, vitamins, suitable precursors, and the like may be included in the medium. The medium or individual components may be added to the medium in a batch or continuous type.

In addition, during the incubation, production of bubbles may be inhibited by using an anti-foaming agent such as fatty acid polyclinic ester.

The incubation of the Streptomyces mutant strain as described above may be performed at 15° C. to 40° C., for example, 20° C. to 35° C. or 25° C. to 30° C. When the incubation of the Streptomyces mutant strain is performed at a temperature of less than 15° C. or more than 40° C., the production amount of the β-agarase DagA enzyme may not be sufficient. In addition, the incubation of the Streptomyces mutant strain as described above may be performed at pH 4.3 to pH 9.5, preferably at pH 5.0 to pH 9.0, more preferably at pH 6.0 to pH 8.0, but is not limited thereto. The incubation pH condition of the Streptomyces mutant strain as described above may be adjusted by adding compounds such as ammonium hydroxide, potassium hydroxide, ammonia, phosphoric acid, and sulfuric acid to the medium of the Streptomyces mutant strain. When the incubation pH condition of the Streptomyces mutant strain is out of the above range, it is difficult to grow the Streptomyces mutant strain, so that it may not be easy to express the β-agarase DagA enzyme.

The β-agarase DagA enzyme over-expressed through the incubation of the Streptomyces mutant strain as described above may be isolated and purified.

The isolation of the β-agarase DagA enzyme may be performed through a protein isolation method commonly performed in the art, such as centrifugation and filtration. In addition, the DagA enzyme isolated by the above method may be purified by a conventional purification method, for example, methods such as salting out (e.g., ammonium sulfate precipitation, sodium phosphate precipitation), solvent precipitation (protein fraction precipitation using acetone, ethanol, etc.), dialysis, gel filtration, ion exchange, chromatography such as reverse-phase column chromatography, and ultrafiltration alone or in combination.

As described above, the β-agarase DagA enzyme obtained from the Streptomyces mutant strain may be used to produce neoagarohexaose or neoagarotetraose in an in vitro reaction.

3. Method of Producing Neoagarohexaose or Neoagarotetraose In Vivo

Another aspect of the present invention provides a method for producing neoagarohexaose or neoagarotetraose in vivo.

The method for producing neoagarohexaose or neoagarotetraose in vivo comprises using a strain in which the activities of both the sco3487 gene and the sco3485 gene are reduced or lost, in the strains described in the item of “1. β-agarase DagA over-producing Streptomyces mutant strain and method for producing the same” above and incubating the strain in a medium containing agar.

As described above, in the strain in which the activities of both the sco3487 gene and the sco3485 gene are reduced or lost, the β-agarase DagA enzyme is expressed at a very high level. At the same time, since the activity of the β-agarase DagB enzyme is reduced or lost, when the strain is incubated in the medium containing the agar, the neoagarohexaose or neoagarotetraose produced by the β-agarase DagA enzyme is not converted into neoagarobiose, but accumulated as it is. Therefore, as described above, the Streptomyces mutant strain in which the activities of both the sco3487 gene and the sco3485 gene are reduced or lost, may produce neoagarohexaose or neoagarotetraose in a culture broth without a separate β-agarase DagA enzyme isolation process.

In addition, the method for producing neoagarohexaose or neoagarotetraose in vivo of the present invention may further comprise purifying neoagarohexaose or neoagarotetraose from a culture broth in which the strain is incubated.

The purification of the neoagarohexaose or neoagarotetraose may be performed by general isolation and purification methods known in the art.

Hereinafter, the present invention will be described in detail by Examples and Test Examples.

However, the following Examples and Test Examples are just illustrative of the present invention, and the contents of the present invention are not limited to the following Examples and Test Examples.

### [Example 1] Preparation of Streptomyces Mutant Strain with Knocked-Down Sco3485 Gene

[1-1] Preparation of Vector for Knocked-Down Sco3485 Gene

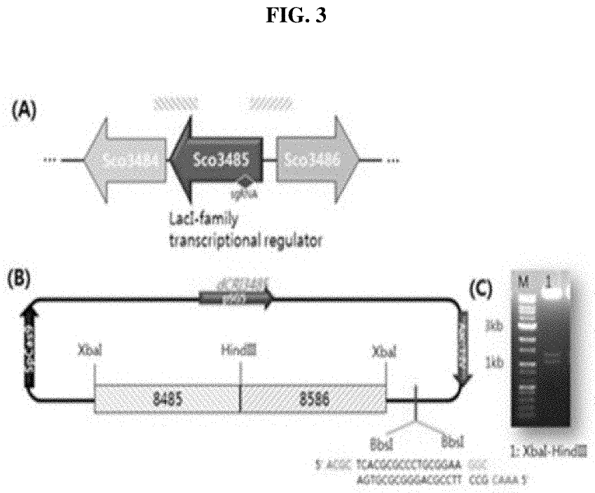

In order to knock down a sco3485 gene in a Streptomyces strain, a vector was prepared in the form as illustrated in (B) of FIG. 3.

First, a sgRNA capable of complementary binding to a part of a nucleic acid sequence of the sco3485 gene to be knocked down, was designed. In order to be able to express the designed sgRNA, two single-stranded DNA oligomers having nucleic acid sequences as illustrated in Table 1 below were prepared, and then these two DNA oligomers were annealed to each other to obtain a double-stranded nucleic acid sequence. Then, the double-stranded nucleic acid sequence annealed as described above was inserted into a BbsI restriction enzyme site of a pCRISPomyces-2 vector (Addgene, USA).

TABLE 1

| Name | Direction | Sequence (5′ → 3′) | SEQ ID NO. |
|---|---|---|---|
| sco3485 | Forward | AAACGCCTTCCGCAGGGCGCGTGA | 2 |
| sgRNA | Backward | ACGCTCACGCGCCCTGCGGAAGGC | 3 |

In addition, when a part of the sco3485 gene targeted by the sgRNA was cleaved by CAS9 and then restored by a homology directed repair (HDR) method, in order to delete the part of the sco3485 gene, the nucleic acid sequence serving as a template of the homologous recombination was prepared and inserted into a pCRISPomyces-2 vector together. The nucleic acid sequence serving as the template of the recombination was prepared by preparing a nucleic acid sequence (see a diagonal blue portion in (A) of FIG. 3) of 1291 bp located upstream of the part targeted by the sgRNA and a nucleic acid sequence (see a diagonal red portion in (A) of FIG. 3) of 1055 bp located downstream thereof in the nucleic acid sequence of the sco3485 gene through PCR reaction using primers disclosed in Table 2 below and then inserting these two PCR products into a XbaI restriction enzyme site of a pCRISPomyces-2 vector. At this time, the nucleic acid sequence serving as the template of the homologous recombination was prepared to include a gap of 68 bp in the sco3485 gene.

TABLE 2

| | | | | SEQ |
|---|---|---|---|---|
| | | | | ID |
| Gene | Location | Name | Sequence (5′ → 3′) | NO. |
| sco3485 | Left | 8586- | TCTAGAACGCGAACTTCGAGCCA | 4 |
| | | XbaI | CCGG | |
| | | 8586- | AAGCTTCGCCGAGGACGCAGCAG | 5 |
| | | HindIII | GGGAC | |
| | Right | 8485- | AAGCTTCGACAGATCAGGGATGA | 6 |
| | | HindIII | GCAGCG | |
| | | 8485- | TCTAGAGCCGTGCGTCGGCAGCG | 7 |
| | | XbaI | G | |

Through the above process, a vector (hereinafter referred to as a ‘dCRI3485 vector’) was prepared to have a map as illustrated in (B) of FIG. 3 in which an sgRNA capable of complementarily binding to a part of the nucleic acid sequence of the sco3485 gene and a nucleic acid sequence serving as a template of the homologous recombination of the sco3485 gene are inserted into a BbsI restriction enzyme site and a XbaI restriction enzyme site of the pCRISPomyces-2 vector, respectively.

[1-2] Preparation of Streptomyces Mutant Strain with Knocked-Down Sco3485 Gene

The dCRI3485 vector prepared in Example [1-1] above was transgened into E. coli ET12567 (puz 8002) to prepare a transgenic E. coli. Thereafter, the transgenic E. coli was conjugated with Streptomyces coelicolor to induce the production of a conjugant. The conjugants were streaked on a medium containing apramycin and nalidixic acid to obtain a conjugant from which E. coli was removed.

The conjugant was incubated at 41° C. for 5 days using a R2YE medium without antibiotics. Thereafter, the conjugant in the medium was spreaded on a plate containing R2YE to form single colonies. The single colonies were duplicated streaking in both R2YE plate and R2YE containing apramycin, respectively, and then only single colonies grown in a medium without apramycin were selected.

In order to finally selection only the transgenic conjugant among the screened single colonies, a PCR reaction for the sco3485 gene was performed using the chromosomal DNA (gDNA) isolated from the selected colonies as a template by using primers shown in Table 3 below. Thereafter, the PCR product was subjected to electrophoresis on a 1% (W/v) agarose gel, and colonies in which the size of the PCR product was reduced by 68 bp compared to a wild type were finally selected.

TABLE 3

| Target | | | SEQ |
|---|---|---|---|
| Gene | Direction | Primer Sequence (5′ → 3′) | ID NO. |
| sco3485 | Forward | ATGCCCCCTGTCATGAAGGTGG | 8 |
| | Backward | GACGACACCACGCTCCGGATCA | 9 |

In addition, in order to confirm whether 68 bp of the sco3485 gene was deleted from the selected strain, as illustrated in FIG. 4, base sequencing was performed on the gene of the finally selected mutant strain. As a result, as illustrated in (C) of FIG. 4, it was confirmed that about 64 bp of the sco3485 gene of the finally selected mutant strain was deleted. The mutant strain confirmed above is named CRI3485 strain.

### [Example 2] Preparation of Streptomyces Mutant Strain with Knocked-Down Sco3487 Gene

[2-1] Preparation of Vector for Knocking-Down Sco3487 Gene

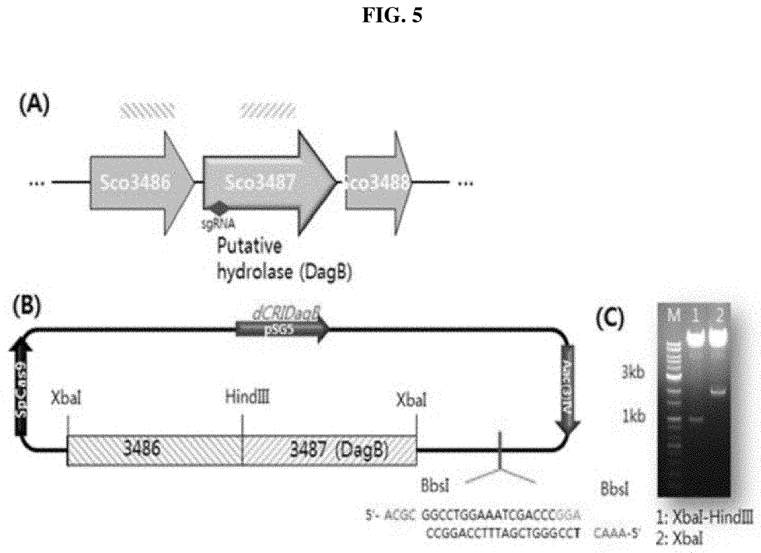

In order to knock-down a sco3487 gene in a Streptomyces strain, a vector was prepared in the form as illustrated in (B) of FIG. 5.

The preparation of the vector was performed in the same manner as in Example [1-1]. However, a nucleic acid sequence was prepared to express a sgRNA capable of complementarily binding to a part of the nucleic acid sequence of the sco3487 gene to be knocked-down using two single-stranded DNA oligomers having nucleic acid sequences shown in Table 4 below. When a part of the sco3487 gene targeted by the sgRNA was cleaved by CAS9 and then restored by a homology directed repair (HDR) method, a nucleic acid sequence as a template of recombination was prepared by preparing a nucleic acid sequence (see a diagonal blue portion in (A) of FIG. 5) of 956 bp located upstream of the part targeted by the sgRNA and a nucleic acid sequence (see a diagonal red portion in (A) of FIG. 5) of 955 bp located downstream thereof in the nucleic acid sequence of the sco3487 gene through PCR reaction using primers disclosed in Table 5 below so that a part of the sco3487 gene was deleted. At this time, the nucleic acid sequence serving as the template of the recombination was prepared to include a gap of 28 bp in the sco3487 gene.

TABLE 4

| | | | SEQ |
|---|---|---|---|
| Name | Direction | Sequence (5′ → 3′) | ID NO. |
| sco3487 | Forward | ACGCGGCCTGGAAATCGACCCGGA | 11 |
| sgRNA | Backward | AAACTCCGGGTCGATTTCCAGGCC | 12 |

TABLE 5

| | | | | SEQ |
|---|---|---|---|---|
| | | | | ID |
| Gene | Location | Name | Sequence (5′ → 3′) | NO. |
| sco3487 | Left | 86-Xba | TCTAGAACGTGCGTCCCGAGA | 13 |
| sgRNA | | | TGGACATCGCGCG | |
| | | 86-Hind | AAGCTTCTTGTCGCCGCGACC | 14 |
| | | | GACG | |
| | Right | dagB- | AAGCTTACTACTCCTCGTTCT | 15 |
| | | Hind | CCGTACGCCCCGAG | |
| | | dagB- | TCTAGAAGCTGTAGGTCTCGC | 16 |
| | | Xba | CCTGCGCG | |

Through the above process, a vector (hereinafter referred to as a ‘dCRIDb vector’) was prepared to have a map as illustrated in (B) of FIG. 5 in which an sgRNA capable of complementarily binding to a part of the nucleic acid sequence of the sco3487 gene and a nucleic acid sequence serving as a template of the recombination of the sco3487 gene were inserted into a BbsI restriction enzyme site and a XbaI restriction enzyme site of the pCRISPomyces-2 vector, respectively.

[2-2] Preparation of Streptomyces Mutant Strain with Knocked-Down Sco3487 Gene

Using the dCRIDb vector prepared in Example [2-1], the sco3487 gene was knocked-down in the Streptomyces strain in the same manner as in Example [1-2]. However, primers for the sco3487 gene used to finally selected colonies used nucleic acid sequences shown in Table 6 below.

TABLE 6

| Target | | | SEQ |
|---|---|---|---|
| Gene | Direction | Primer Sequence (5′ → 3′) | ID NO. |
| sco3487 | Forward | TTGCACCACTCCGCCGCCGC | 17 |
| sgRNA | Backward | AGTACGTGCCACCGCCGCCG | 18 |

Colonies were screened in which a size of the PCR product obtained by performing a PCR reaction for the sco3487 gene using a chromosomal DNA isolated from the selected mutant as a template was reduced by 28 bp compared to a wild type. As a result, it was confirmed that as illustrated in (C) of FIG. 6, the sco3485 gene of the finally selected mutant was deleted by 28 bp. The selected mutant confirmed above is named CRIDb strain.

### [Example 3] Preparation of Streptomyces Mutant Strain with Knocked-Down Both Sco3485 and Sco3487 Genes

In the CRIDb strain prepared in Example [2-2], the dCRI3485 vector prepared in Example [1-1] was transgened in the same manner as in Example [1-2] to prepare a Streptomyces mutant strain in which both the sco3487 gene and the sco3485 gene were knocked-down. However, primers for the sco3485 gene and the sco3487 gene used to finally screen colonies used nucleic acid sequences shown in Tables 3 and 6 above.

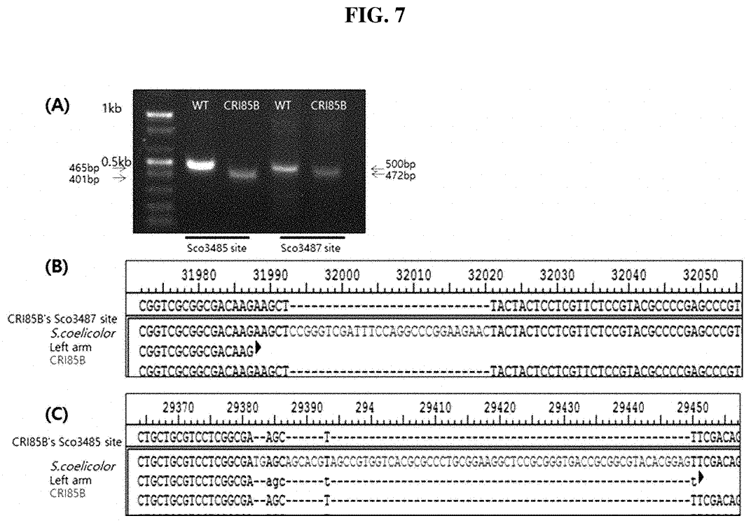

Colonies were finally screened in which sizes of PCR products obtained by performing a PCR reaction for the sco3485 gene and the sco3487 gene using a chromosomal DNA isolated from the selected mutant as a template were reduced by 64 bp and 28 bp compared to a wild type, respectively. As a result, it was confirmed that as illustrated in FIG. 7, the finally selected mutant was deleted by 64 bp of the sco3485 gene and 28 bp of the sco3487 gene, respectively.

The selected mutant confirmed above is named a CRI85B strain.

### [Test Example 1] Reaction with Agarose Using Supernatant from Culture Medium of CRIDb Strain

It was confirmed how agarose was converted by enzymes produced in a CRIDb strain in which the sco3487 gene was knocked-down.

First, a wild-type Streptomyces strain and the CRIDb strain were incubated for 5 days in a medium containing agar. The culture broth was subjected to 70% ammonium sulphate precipitation and the extracellular precipitate then was diluted to a concentration of 1 mg/ml. The protein concentrate reacted with a solution of 0.2% agarose (dissolved in 20 mM Tris-HCl (pH 7.0)) as a substrate at 40° C. for 1 hour and 18 hours, respectively. After the reaction was stopped by boiling the reaction mixtures for 10 minutes, the product was applied to a thin-layer chromatography (TLC) plate.

As a result, as illustrated in FIG. 8, a protein isolated from the wild-type Streptomyces strain degraded agarose to produce neoagarobiose, NA4, and NA6. On the other hand, the protein isolated from the CRIDb strain degraded agarose to produce only NA4 and NA6.

Through the above results, it can be seen that the CRIDb strain according to the present invention prevents the β-agarase DagB enzyme encoded by the gene from exhibiting its function because the Sco3487 gene is knocked-down, so that the intermediates NA6 and NA4 are not degraded into neoagarobiose as a final product, but accumulated.

### [Test Example 2] Phenotypic Analysis of CRIDb Strain, CRI3485 Strain, and CRI85B Strain

A Phenotypic analysis was performed to identify the phenotypes according to the agarases production of each CRIDb strain, CRI3485 strain, and CRI85B strain. The strains cultured on minimal agar media (MM), MM containing agarose (MA) and galactose (MG) as carbon sources, respectively, and then used to measure the protein activity through a zymogram assay.

To perform the zymogram analysis, first, the three strains were spotted onto each medium plates with the same size, and then incubated at 28° C. for 48 to 120 hours. Thereafter, a Lugol's solution containing 25 g of iodine and 50 g of potassium iodine per 1 L was smeared on a plate, and a shape of a ring formed thereby was confirmed.

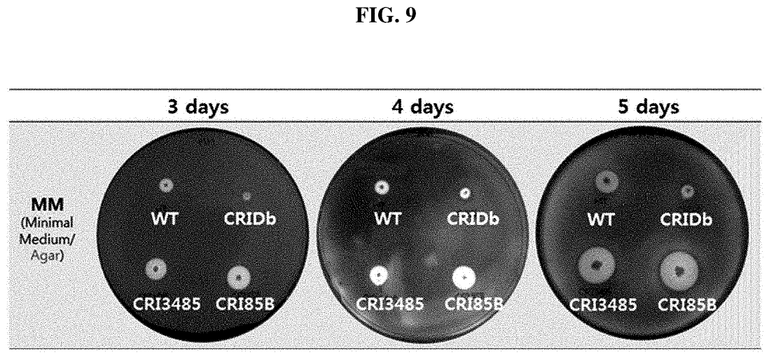

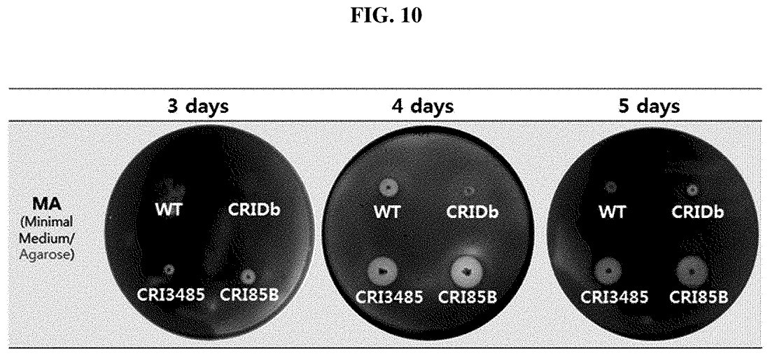

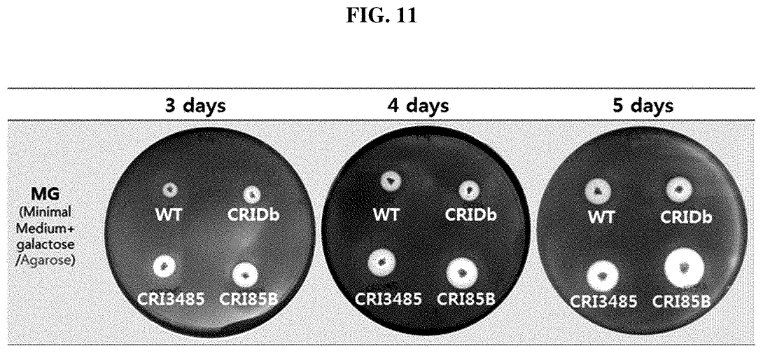

As a result, as illustrated in FIGS. 9 to 11, in a minimal agar plates (MM), agarose (MA), and galactose (MG) as a carbon source, compared to the wild-type strain, a clear halo corresponding to a degradation zone of agar is larger according to the agarase production of the CRI3485 strain and the CRI85B strain. In addition, the CRIDb strain showed a clear halo smaller or similar to that of the wild type in the all kinds of medium.

Through the above results, it can be seen that the protein encoded by the sco3485 gene may inhibit the expression of the β-agarase DagA enzyme and the genes encoding the protein participating in the process of degrading agarose. In addition, since a difference in the clear zone is clear from the beginning time of culture, it can be seen that the product of the gene will acts as a regulator to suppress early gene expression without external factors. Even if the sco3485 gene of the CRIDb strain is knocked-down, slowly growth of the CRIDb strain compared to other strains means that the DagB plays an important role in growth when using an agar as its unique carbon sources. It can be seen that such a phenomenon is more evident in the galactose medium (MG), and the expression of the β-agarase DagA enzyme is remarkable.

### [Test Example 3] Expression Analysis of β-Agarase DagA Enzyme of CRIDb Strain, CRI3485 Strain, and CRI85B Strain

The expression level of secreted β-agarase DagA enzyme in the supernatant on the culture medium from the CRIDb, CRI3485, and CRI85B strain was quantified by using the Western blot analysis.

The strains were cultured in RSM3+AO broth, which is a liquid medium containing agar for 3 and 5 days for agarase production. Cultured samples were used for measuring DagA agarase expression. The western blot analysis was performed using a specific the β-agarase DagA antibody, and the results thereof were shown in FIG. 12.

As shown in FIG. 12, on 3 days, the expression levels of the β-agarase DagA enzyme were 0.3 times for the CRIDb strain, 1.6 times for the CRI3485 strain, and 4.6 times for the CRI85B strain larger than that of the wild type strain, respectively. On 5 days, the expression levels of the β-agarase DagA enzyme were 0.5 times for the CRIDb strain, 4.9 times for the CRI3485 strain, and 8.1 times for the CRI85B strain larger than that of the wild type strain, respectively.

Through the above results, it can be seen that when the sco3485 gene is knocked-down in the Streptomyces strain, the expression of the β-agarase DagA enzyme is increased, and when the soc3487 gene encoding the β-agarase DagB enzyme is knocked-down together, the expression of the β-agarase DagA enzyme is further increased.

As described above, the present invention has been described in detail only with respect to the described embodiments, but it will be apparent to those skilled in the art that various modifications and changes can be made within the scope of the technical idea of the present invention, and it is natural that such modifications and changes belong to the appended claims.

1. A Streptomyces mutant strain over-expressing a β-agarase DagA enzyme in which the activity of a sco3485 gene is reduced or lost.

2. The Streptomyces mutant strain of claim 1, wherein the sco3485 gene has a nucleic acid sequence of SEQ ID NO: 1.

3. The Streptomyces mutant strain of claim 1, wherein the Streptomyces mutant is a strain in which the activity of a sco3487 gene is further reduced or lost.

4. The Streptomyces mutant strain of claim 3, wherein the sco3487 gene has a nucleic acid sequence of SEQ ID NO: 10.

5. A method for producing a Streptomyces mutant strain over-expressing a β-agarase DagA enzyme comprising reducing or losing the activity of a sco3485 gene in a Streptomyces strain.

6. The method for producing the Streptomyces mutant strain of claim 5, further comprising:
reducing or losing the activity of a sco3487 gene.

7. The method for producing the Streptomyces mutant strain of claim 6, wherein the activities of the sco3485 gene and the sco3487 gene are reduced or lost sequentially or simultaneously.

8. A method for producing a β-agarase DagA enzyme comprising:
incubating the Streptomyces mutant strain of claim 1; and
isolating the β-agarase DagA enzyme from the incubated Streptomyces mutant strain.

9. A method for producing neoagarohexaose or neoagarotetraose in vivo comprising incubating the Streptomyces mutant strain of claim 3 in a medium containing agar.

10. The method for producing the neoagarohexaose or neoagarotetraose in vivo of claim 9, further comprising:
isolating or purifying the neoagarohexaose or neoagarotetraose from the incubated medium.


SEQUENCE LISTING (18 sequences)

SEQ ID NO: 1 - DNA, 1050 bp, Streptomyces coelicolor
atgacatcggaggcaggggcgggtcaggcgaaagcgcttccggcgcgagcgcggctcgtcgacgtggcgaagcgcgcgggggtgaccaagtccgttgtctcacggatcctgaacgacgacaccacgctccggatcaggcccgagacgcgtgagcgcgtcctcgctgtcgccgcggaactcgggtatcggcccaacgtcacggcgcgcgcgctgtcggtgtcgcggaccagggcgttcgcgctgctcatccctgatctgtcgaactccgtgtacgccgcggtcacccgcggagccttccgcagggcgcgtgaccacggctacgtgctgctcatcgccgaggacgcagcaggggacgacgacgcggcctatgcggagctggtgacggccgggcgggtggacggtctcctcgtcgcgtcgtcccggccggagcaccccctggtcgagcgcctcctcgccgatcgcggtgggctggcgcacgtctttctcaaccgggaggtccaggggtccaaccgtaacgtcggcatggatatgcagggggcgagcgccctggtcgtcgaccaccttcatgacagggggcatcggcatatcggaatggtgacgggtcccctggacctcgctccggcccggtcccggctggacgggtttcagggcaggctgcgcgagctgggcctggacgacaccgcccacttcgccggaaccttcagcgaggccggcggatgcgcggcggcacatggactgcttgacgcgcggccggagctgaccgcgctgtacgccagcacgttcccgcaggccgtcggggtgatgaaggcggtgcgtgaccgagggtggaaggtgcccgccgacgtgtcgctgatcgcgtacgacgacctccccatggcggagttcctcgatcctcccctcaccacgctcgccatgccgctggcgacgctgggagccgcggccgtcgacgctctcgtcgaccagatagagggccgtgtgatcggcagccgtcaggtgctgggggggtacgagatcgtggagcgggcctcggtcgccgttccgcgagtctga

SEQ ID NO: 2 - DNA, 24 bp, Artificial Sequence
  sco3585 sgRNA
aaacgccttccgcagggcgcgtga

SEQ ID NO: 3 - DNA, 24 bp, Artificial Sequence
  sco3585 sgRNA
acgctcacgcgccctgcggaaggc

SEQ ID NO: 4 - DNA, 27 bp, Artificial Sequence
  sco3485 HDR XbaI left
tctagaacgcgaacttcgagccaccgg

SEQ ID NO: 5 - DNA, 28 bp, Artificial Sequence
  sco3485 HDR XbaI left
aagcttcgccgaggacgcagcaggggac

SEQ ID NO: 6 - DNA, 29 bp, Artificial Sequence
  sco3485 HDR HindIII Right
aagcttcgacagatcagggatgagcagcg

SEQ ID NO: 7 - DNA, 24 bp, Artificial Sequence
  sco3485 HDR XbaI  Right
tctagagccgtgcgtcggcagcgg

SEQ ID NO: 8 - DNA, 22 bp, Artificial Sequence
  sco3485 forward primer
atgccccctgtcatgaaggtgg

SEQ ID NO: 9 - DNA, 22 bp, Artificial Sequence
  sco3485 reverse primer
gacgacaccacgctccggatca

SEQ ID NO: 10 - DNA, 2397 bp, Streptomyces coelicolor
atgaccgtgcacaagcgcgcttgcaccactccgccgccgcgagccagcaggtcgttccgcgtgaggtggcctgtcctgatagcggccgcctgcgccgggttagtcctggcgaccaccagccctccggccgtcgcggccggcgctcatgacctcggcgacgagaccatgctctacgacttccaggacggcctggtaccggccgaggtcggcccgtaccaggcgaagacgacaatcgtcggtcgcggcgacaagaagctccgggtcgatttccaggcccggaagaactactactcctcgttctccgtacgccccgagcccgtgtggaactggtcggcggaggagtccgagtcgctcggcatcgcgatggagctcacgaacccgagcgaccgctccgtccagctcacgatcgatctggagagctcgaccggcgtcgccacccgcagtgtcaacgtcccggccggcggcggtggcacgtactacttcgacgtcgacagccccgcgctccaccgcgacaccgggctgcgcgccgatccgtcctggctcgcggacaaggacgtcacctctgcggtctggatgtggggctccaaggagacggacacgagccgcatcagccagctgaacttctacgtcgccggcctgctgcacgaccggtcggtcatcgtggacgacatccgcgtcgtccgtgacgcgccggcagaccccgattacctcaagggcctggtcgacgccttcggtcagaacaacaaggtcgactacaagggaaaggtctccaggacgtcggagattcttcggcagcgcgctgccgaagccaaggaccttcgcaggcatccggttccggaggaccggtccaggtacggcggctggctgaacggtccacgactcgaggcgacgggcaacttccgcgtggagaagtaccaggggcggtggaccctggtggaccctgacggctacctgttcttctcaaccggcatcgacaacgcccgcatgttcgactccccaaccacgacgggttacgacttcgaccatgacgcgatccaggagctgccgccccccagcctgacggccggcggccccgaggacctcaaccgcgtccagaagtcggcgctgccgacccgcacgaagatgtccgaaacccgcgccgacctcttcagcaagttgcccaagtaccgcacccgcgcgggcgagggcttcggttacgcccccgacaccctggccggtcccgtcgcgcagggcgagacctacagcttctacaaggcgaacgtcgcccggaagtaccccggcagcaactacatggagcggtggcgggacaacacggtcgaccggatgctcagctggggcttcacctccttcggcaactggaccgacccggagatgtacgacaacgaccgtatcccgtacttcgcccacggctggatcaagggcgacttcaagacggtgagcaccggccaggactactggggcccgatgccggacccgttcgaccccgcgttctccgacgccgcagccagaaccgcgcgagcagtcgccgacgaggtcgcggacagcccgttggcgatcggcgtattcatggacaacgaactgagctggggcaacgccggcagtttcagcacccgttacggcgtcgtcatcgacaccatgtcacgtgacgcggcagagagccccaccaagtcggcgttctccgacgaactggaggagaagtacgggaccatcgacgctctcaacgccgcgtggcagacgacagttccgtcatgggaggcactccgtagcggcagtgccgacctcggctccgacgagaccgcgaaggagtccgactactccgcgctcatgacgctctacgccactcagtacttcaagacggtcgacgccgagctcgacaaggtcatgccggaccatctctacgcgggttcgaggttcgccagctggggccgcacaccggaggtcgtcgaggcagcgagcaagtacgtcgacatcatgagctacaacgagtaccgcgagggactgcacccgagcgagtgggcgtttctcgaagagctcgacaagcccagcctcatcggtgagttccacatgggaacgaccactaccgggcagccgcatccgggtctcgtctcggcgggaacgcaggccgagcgggcacggatgtacgccgagtacatggaacagctcatcgacaacccgtacatggtgggcggccactggttccagtatgccgactcgcccgtgactggcagagcactcgacggggagaactacaacattggcttcgtctccgtcacggaccgtccctacccggagatcgtcgccgctgcccgcgacgtgaaccagcgtctctatgaccgccgatacggcaacctggccacggccgagggacattacaccggtcggcgttcagcggagtag

SEQ ID NO: 11 - DNA, 24 bp, Artificial Sequence
  sco3487 sgRNA
acgcggcctggaaatcgacccgga

SEQ ID NO: 12 - DNA, 24 bp, Artificial Sequence
  sco3487 sgRNA
aaactccgggtcgatttccaggcc

SEQ ID NO: 13 - DNA, 34 bp, Artificial Sequence
  sco3487 HDR XbaI left
tctagaacgtgcgtcccgagatggacatcgcgcg

SEQ ID NO: 14 - DNA, 25 bp, Artificial Sequence
  sco3487 HDR HindIII left
aagcttcttgtcgccgcgaccgacg

SEQ ID NO: 15 - DNA, 35 bp, Artificial Sequence
  sco3487 HDR HindIII Right
aagcttactactcctcgttctccgtacgccccgag

SEQ ID NO: 16 - DNA, 29 bp, Artificial Sequence
  sco3487 HDR XbaI Right
tctagaagctgtaggtctcgccctgcgcg

SEQ ID NO: 17 - DNA, 20 bp, Artificial Sequence
  sco3487 forward primer
ttgcaccactccgccgccgc

SEQ ID NO: 18 - DNA, 20 bp, Artificial Sequence
  sco3487 reverse primer
agtacgtgccaccgccgccg